(19) United States
(12) Patent Application Publication
(10) Pub. No.: US 2010/0331838 A1
(43) Pub. Date: Dec. 30, 2010

(54) TRANSMURALITY CLAMP SYSTEMS AND METHODS

(75) Inventors: Tamer Ibrahim, Pleasant Hill, CA (US); David K. Swanson, Campbell, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ESTECH, Inc. (Endoscopic Technologies, Inc.), San Ramon, CA (US)

(21) Appl. No.: 12/822,616

(22) Filed: Jun. 24, 2010

Related U.S. Application Data
(60) Provisional application No. 61/220,414, filed on Jun. 25, 2009.

(51) Int. Cl. A61B 17/28; A61B 18/18; A61B 5/00

(52) U.S. Cl. 606/52; 600/301

(57) ABSTRACT

Tissue clamp systems and methods assess the transmurality of a lesion created by ablation. Exemplary clamp systems for ablating tissue include a first jaw, an ablation element mounted on the first jaw, a second jaw, and a sensing element mounted on the second jaw. The sensing element can be configured to assess transmurality of a lesion created by the ablation element. Systems may also include a connection mechanism that connects the ablation element and the sensing element to a connector for connecting the ablation element and the sensing element to an ablation system.

### CROSS-REFERENCES TO RELATED APPLICATIONS

This application is a nonprovisional of, and claims the benefit of the filing date of U.S. Provisional Patent Application No. 61/220,414 filed Jun. 25, 2009, entitled “Transmurality Clamp Systems and Methods,” the entire disclosure of which is incorporated herein by reference for all purposes.

### BACKGROUND OF THE INVENTION

Embodiments of the present invention relate in general to tissue ablation systems and methods. Particular embodiments related to clamp systems and methods for assessing the transmurality of a lesion created by ablation.

There are many instances where it is beneficial to perform a therapeutic intervention in a patient, using a system that is inserted within the patient's body. One exemplary therapeutic intervention involves the formation of therapeutic lesions in the patient's heart tissue to treat cardiac conditions such as atrial fibrillation, atrial flutter, and arrhythmia. Therapeutic lesions may also be used to treat conditions in other regions of the body including, but not limited to, the prostate, liver, brain, gall bladder, uterus, and other solid organs. Typically, the lesions are formed by ablating tissue with one or more electrodes. Electromagnetic radio frequency (“RF”) energy applied by the electrode heats and eventually kills or ablates the tissue to form a lesion. During the ablation of soft tissue (e.g. tissue other than blood, bone and connective tissue), tissue coagulation occurs, which leads to tissue death. Thus, references to the ablation of soft tissue are typically references to soft tissue coagulation. “Tissue coagulation” can refer to the process of cross linking proteins in tissue to cause the tissue to jell. In soft tissue, it is the fluid within the tissue cell membranes that jells to kill the cells, thereby killing the tissue. Depending on the procedure, a variety of different electrophysiology devices may be used to position one or more electrodes at the target location. Electrodes can be connected to power supply lines and, in some instances, the power to the electrodes can be controlled on an electrode-by-electrode basis. Examples of electrophysiology devices include catheters, surgical probes, and clamps.

Currently known surgical probes which can be used to create lesions often include a handle, a relatively short shaft that is from 4 inches to 18 inches in length and either rigid or relatively stiff, and a distal section that is from 1 inch to 10 inches in length and either malleable or somewhat flexible. One or more electrodes are carried by the distal section. Surgical probes are used in epicardial and endocardial procedures, including open heart procedures and minimally invasive procedures where access to the heart is obtained via a thoracotomy, thoracostomy or median sternotomy. Exemplary surgical probes are disclosed in U.S. Pat. No. 6,142,994, the content of which is incorporated herein by reference.

Clamps, which have a pair of opposable clamp members that may be used to hold a bodily structure or a portion thereof, are used in many types surgical procedures. Lesion creating electrodes have also been secured to certain types of clamps. Examples of clamps which carry lesion creating electrodes are discussed in U.S. Pat. No. 6,142,994, and U.S. Patent Publication Nos. 2003/0158549, 2004/0059325, and 2004/024175, the contents of which are incorporated herein by reference. Such clamps can be useful when the physician intends to position electrodes on opposite sides of a body structure in a bipolar arrangement.

Although these and other proposed treatment devices and methods may provide real benefits to patients in need thereof, still further advances would be desirable. For example, there continues to be a need for improved ablation systems and methods that can be used by surgeons to treat patient tissue or anatomical features having various sizes, shapes, densities, and the like. Embodiments of the present invention provide solutions that address the problems which may be associated with known techniques, and hence provide answers to at least some of these outstanding needs.

### BRIEF SUMMARY OF THE INVENTION

Systems for ablating tissue can be used with any preferred surgical access technique, including without limitation sternotomy and thoracotomy procedures. Exemplary tissue ablation systems may include a clamp structure for clamping single or double wall thickness tissue. According to some embodiments, an ablation system may include a unipolar ablation clamp. For example, a first jaw of the clamp can have include an active energy delivery mechanism which may incorporate any of a variety of energy sources, including radiofrequency (RF), ultrasound, cryothermy, microwave, laser, and the like. Relatedly, the energy delivery mechanism may incorporate any of a variety of transmission mechanisms, such as electrodes, transducers, antennas, and the like.

A tissue ablation system can include a clamp having a first jaw and a second jaw. The first jaw may have an active energy delivery mechanism that includes a temperature sensor as a means of controlling energy delivery to ensure target tissue reaches target temperature. A second, opposing jaw can have either a continuous element or series of one or more discrete temperature sensing elements for same purpose. Temperature sensors can provide feedback to a energy delivery source that delivers energy until sensors on an inactive jaw register that the target temperature has been reached or a predefined timepoint, in the event the temperature endpoint is not reached within that timeframe. Temperature sensing elements may be complimented by pacing and sensing elements to determine lag in conduction time across lesion. Clamp may include visualization and delivery system including scopes with protective lenses and introducers with stylets, sheaths, and or magnets.

In one aspect, embodiments of the present invention encompass clamp assembly systems and methods for ablating tissue of a human patient. An exemplary clamp assembly for ablating tissue may include a first jaw, an ablation element mounted on the first jaw, a second jaw, and a sensing element mounted on the second jaw. The sensing element can be configured to assess transmurality of a lesion created by the ablation element. The clamp assembly may also include a connection mechanism that connects the ablation element and the sensing element to a connector for connecting the ablation element and the sensing element to an ablation system. In some cases, the connection mechanism includes one or more wires. In some cases, the sensing element includes a temperature sensor. Optionally, the sensing element may include a pacing electrode. Relatedly, the sensing element may include an optical sensor. In some cases, the optical sensor detects tissue color change that occurs as tissues are heated to above 60° C. In some cases, the optical sensor detects changes to near-field microwave that occur as tissues temperatures rise or fall. In some cases, a sensing element penetrates a tissue surface of a tissue as the tissue is clamped between the first and second jaws of the clamp assembly. Optionally, the sensing element may include a temperature sensor. In some instances, the sensing element can include a pacing electrode. According to certain embodiments of the present invention, the sensing element includes an electrode that senses tissue impedance. Optionally, the ablation element can be configured to heat tissue using radiofrequency energy. In some instances, the ablation element heats tissue using microwave energy. In some instances, the ablation element heats tissue using ultrasonic energy. According to some embodiments, the ablation element freezes tissue to achieve ablation. A clamp assembly may also include one or more ablation elements coupled with the second jaw.

In another aspect, embodiments of the present invention encompass systems for ablating tissue which include, for example, an ablation clamp having a first jaw, a second jaw, one or more ablation elements mounted on the first jaw, and a sensing element mounted on the second jaw that assesses transmurality of a lesion created by the ablation element. Tissue ablating systems may also include an ablation control system for controlling an ablation process provided by the ablation clamp, and means, such as wires, for connecting the ablating element and the sensing element with the ablation control system. In some cases, the ablation control system can stop the ablation process when the sensing element carried by the second jaw indicates that a transmural lesion has been created. In some cases, the ablation control system can stop the ablation process if a predetermined maximum time limit has been reached.

In a further aspect, embodiments of the present invention encompass a clamp assembly for ablating tissue that includes a first jaw, an ablation element mounted on the first jaw, a second jaw, and a sensing element mounted on the second jaw. The sensing element can include a temperature sensor, a pacing element, or an optical sensor, and can be configured to penetrate a tissue surface of a tissue as the tissue is clamped between the first and second jaws of the clamp assembly. In some cases, the sensing element can be configured to assess transmurality of a lesion created by the ablation element. A clamp assembly may also include a connection mechanism that connects the ablation element and the sensing element to a connector for connecting the ablation element and the sensing element to an ablation system.

### BRIEF DESCRIPTION OF THE DRAWINGS

FIG. 1 shows aspects of a tissue treatment system according to embodiments of the present invention.

FIG. 2 shows aspects of a tissue treatment system according to embodiments of the present invention.

FIGS. 3A and 3B show aspects of a tissue treatment system according to embodiments of the present invention.

FIGS. 4A, 4B, and 4C show aspects of a tissue treatment system according to embodiments of the present invention.

FIG. 5 shows aspects of a tissue treatment system according to embodiments of the present invention.

FIGS. 6A and 6B show aspects of a tissue treatment system according to embodiments of the present invention.

### DETAILED DESCRIPTION OF THE INVENTION

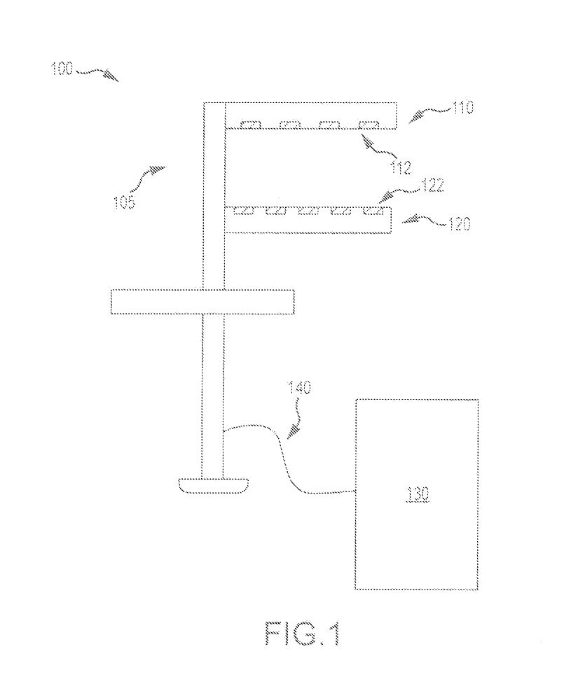

Turning now to the drawings, FIG. 1 shows a system 100 for ablating tissue, according to embodiments of the present invention. System 100 may have a clamp assembly 105 that includes a first jaw 110 and one or more ablation elements 112 mounted on or coupled with first jaw 110. In some cases, ablation elements 112 can be configured to heat tissue using radiofrequency energy. For example, the ablation elements can include active radiofrequency (RF) ablation mechanisms. In some cases, ablation elements 112 may heat tissue using microwave energy. Optionally, ablation elements 112 may heat tissue using ultrasonic energy. Embodiments also provide ablation elements 112 that freeze tissue to achieve ablation.

Clamp assembly 105 may also include a second jaw 120 and one or more sensing elements 122 mounted on or coupled with second jaw 120. Sensing elements 122 can be configured to assess the transmurality of a lesion created by ablation elements 112. Clamp assembly 100 may also include wires connecting the ablation elements 112 and the sensing elements 122 to a connector for connecting the ablation elements 112 and the sensing elements 122 to an ablation system. Sensing elements 122 may in some cases include temperature sensors. Optionally, sensing elements 122 may include pacing electrodes. According to some embodiments, sensing elements 122 include optical sensors. For example, sensing elements 122 may include optical sensors that detect tissue color changes that occur as tissues are heated to above 60° C. Relatedly, sensing elements 122 may include optical sensors that detect changes to near-field microwave that occurs as tissues temperatures rise or fall. In some instances, sensing elements 122 may include electrodes that sense tissue impedance. Sensing elements 122 may be configured to penetrate the tissue surface as the tissue is clamped between the first jaw 110 and the second jaw 120 of clamp assembly 100.

As shown in FIG. 1, system 100 may include an ablation control system for controlling the ablation process provided by the ablation clamp 105. System 100 may further include a means for connecting ablating elements 112 and sensing elements 122 carried by ablation clamp 105 to an ablation control system 130. For example, system 100 may include a connection mechanism 140 that connects ablating elements 112 and sensing elements 122 with ablation control system 130.

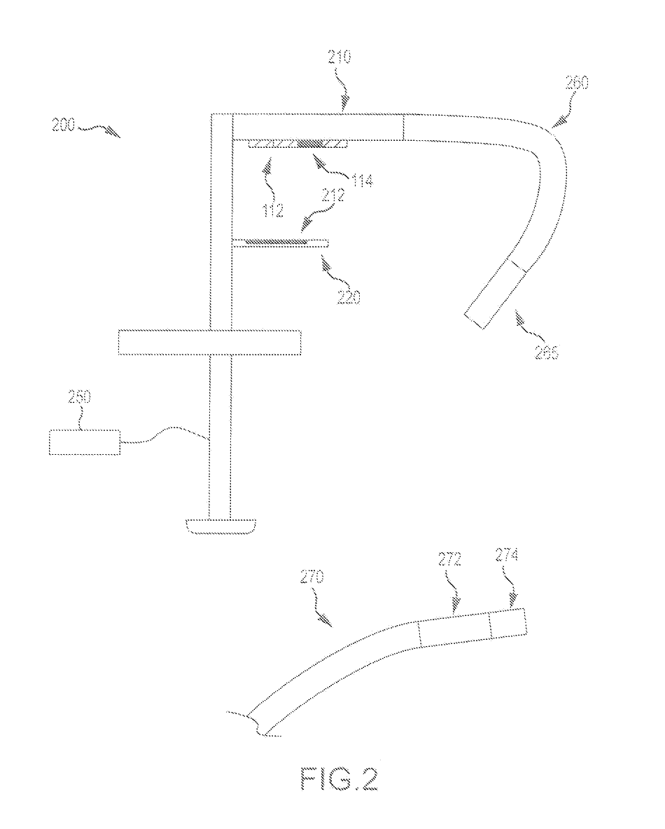

FIG. 2 depicts a system 200 for delivering a tissue ablation treatment to a patient. System 200 includes a clamp assembly 205 having a first jaw 210 and a second, opposing jaw 220. First jaw 210 may have an active energy delivery mechanism 212 that includes a temperature sensor 214 as a means of controlling energy delivery to ensure target tissue reaches target temperature. Second jaw 220 can have either a continuous element 212 or series of one or more discrete temperature sensing elements for same purpose. Temperature sensors can provide feedback to a energy delivery source 250 that delivers energy until sensors on an inactive jaw register that the target temperature has been reached or a predefined timepoint, in the event the temperature endpoint is not reached within that timeframe. Temperature sensing elements may be complimented by pacing and sensing elements to determine lag in conduction time across lesion. Clamp system 200 may incorporate visualization and delivery system elements including scopes with protective lenses and introducers with stylets, sheaths, and or magnets. For example, as shown in FIG. 2, system 200 includes an introducer 260 having a magnet 265, and a scope 270 having a scope lens 272 and a magnet 274.

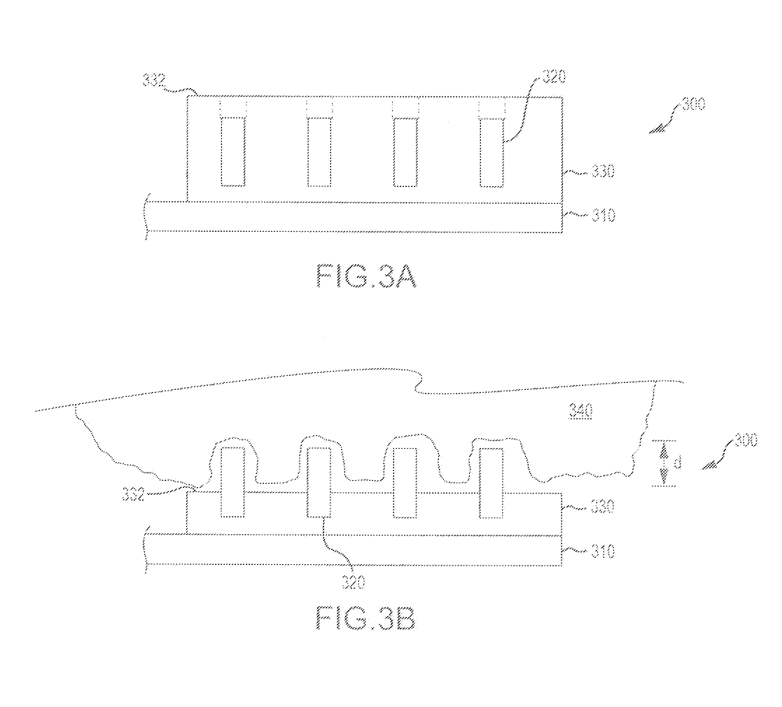

FIGS. 3A and 3B illustrate aspects of a transmurality clamp system according to embodiments of the present invention. As depicted in FIG. 3A, a system can have a clamp jaw 300 which includes a base member 310, one or more penetrating sensors or electrodes 320, and a resilient or compressible structure 330. Clamp jaw 300 may present any number of sensors 320. For example, in some cases, a clamp jaw may present two sensors. In other instances, a clamp jaw may present four sensors. Sensors 320 may include impedance sensors, temperature sensors, and the like. Resilient structure 330 may be constructed of or include a compressible material such as an open cell foam, a closed cell foam, a low durometer silicon rubber, or the like. As shown here, sensors 320 are disposed slightly below or recessed from a contact surface 332 of resilient member 330, when the resilient member is in an uncompressed configuration, such as when little or no pressure is being applied to the resilient member. In contrast, FIG. 3B illustrates a configuration of resilient member 330 when a pressure is applied to contact surface 332, for example when clamp jaw 300 is pressed against a patient tissue 340. Penetrating sensors 320 now protrude into patient tissue 340 to a depth d. In some cases, depth d may vary depending on the compressibility of resilient member 330. Hence, when pressure is applied, contact surface 332 of resilient member 330 retracts so as to expose sensors 320. In some instances, resilient member 330 and sensors 320 are configured so that sensors 320 can extend or penetrate into a patient tissue to a depth d of about 2 mm. Hence, impedance, temperature, or other parameters may be sensed at locations below the tissue surface. In some cases, subsurface sensing can provide a more accurate assessment of a tissue condition, when compared to surface sensing.

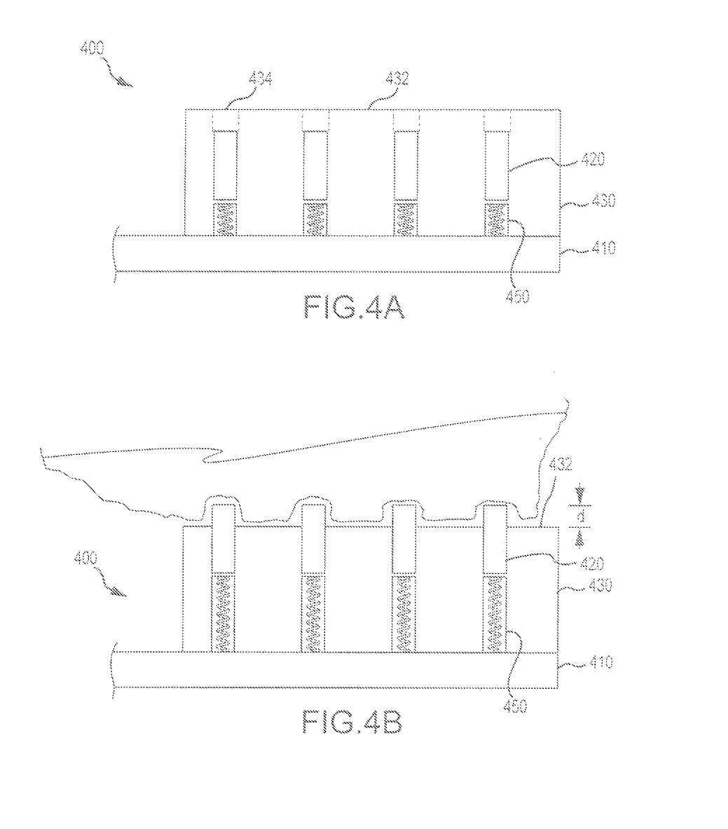

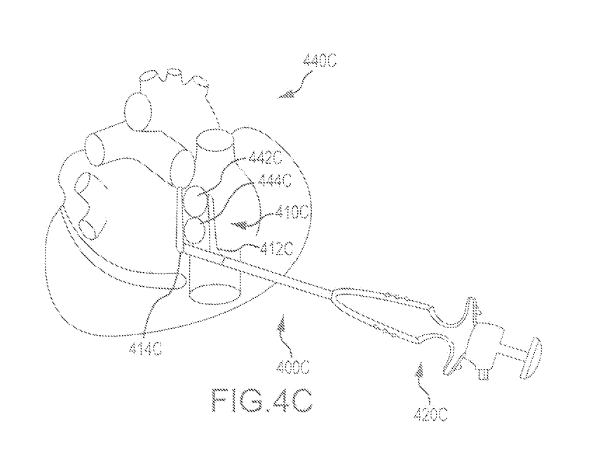

FIGS. 4A and 4B illustrate aspects of a transmurality clamp system according to embodiments of the present invention. As depicted in FIG. 4A, a system can have a clamp jaw 400 which includes a base member 410, one or more penetrating sensors or electrodes 420, a housing element 430, and one or more actuating mechanisms 450. Clamp jaw 400 may present any number of sensors 420. For example, in some cases, a clamp jaw may present two sensors. In other instances, a clamp jaw may present four sensors. Sensors 420 may include impedance sensors, temperature sensors, and the like. Housing element 430 can include channels 434 within which sensors 420 may translate. As shown here, sensors 420 are disposed slightly below or recessed from a contact surface 432 of housing element 430, when the actuating mechanisms are in a retracted configuration. In contrast, FIG. 4B illustrates an extended configuration of actuating mechanisms 450, such that an actuating mechanism 450 is expanded so as to push or expel a penetrating sensor 420 outward from channel 434, and into a patient tissue 440. In some cases, an actuating mechanism 450 may include a spring mechanism. Penetrating sensors 420 now protrude into patient tissue 440 to a depth d. In some cases, depth d may vary depending on the extent to which actuating mechanism 450 is expanded. In some instances, actuating mechanism 450 and sensor 420 are configured so that sensor 420 can extend or penetrate into a patient tissue to a depth d of about 2 mm. Hence, impedance, temperature, or other parameters may be sensed at locations below the tissue surface. In some cases, subsurface sensing can provide a more accurate assessment of a tissue condition, when compared to surface sensing.

FIG. 4C shows a treatment system 400c in a position for performing an ablation or treatment procedure on epicardial tissue of heart 440c. Treatment system 400c includes a clamp assembly 410c having first and second jaw mechanisms 412c, 414c, and can be configured to ablate in a pattern approximating two lines adjacent the right pulmonary veins 442c, 444c. Jaw mechanisms 412c, 414c can be positioned as desired to provide a variety of ablation configurations. Additionally, treatment system 400c may be moved to a variety of positions to ablate multiple patterns in multiple locations on the epicardial tissue.

Treatment system 400c includes a handle or actuator assembly 420c disposed toward a proximal portion of the system. As shown here, first and second jaw mechanisms 412c, 414c, which may include two bipolar ablation clamps, are disposed toward a distal portion of the system. The jaw mechanisms 412c, 414c can be curved or shaped. In some cases, jaw mechanisms 412c, 414c are curved and adjustable. In some cases, a jaw mechanism can be in connectivity with an ablation and monitoring assembly or ESU. During use, the tissue treatment system can be used to contact the cardiac tissue, which can be effectively accomplished for example by the curvature orientation. The curved or contoured shape of the jaw mechanisms can allow the treatment system to be placed on the heart without impinging upon the pulmonary veins. Hence, there is an increased likelihood of ablating tissue of the atrium, as opposed to ablating tissue of the pulmonary veins themselves. Treatment system 400c is well suited for use in surgical methods where access ports are not employed. For example, the treatment system can be inserted into a patient via a 3-4 inch thoracotomy. In use, the jaw mechanisms are placed at or near the ostia, and actuated until the opposing jaw members are approximately 2-5 millimeters apart. This action serves to collapse the atrium near the pulmonary veins. An ablation is performed, and the clamping pressure is released thus allowing the atrium to return to the uncompressed state.

Electrosurgical Unit Operation

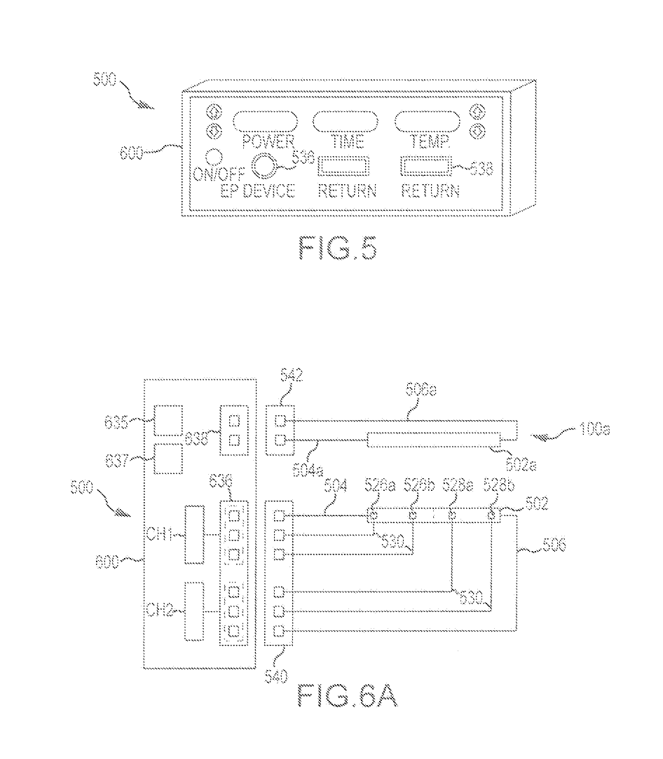

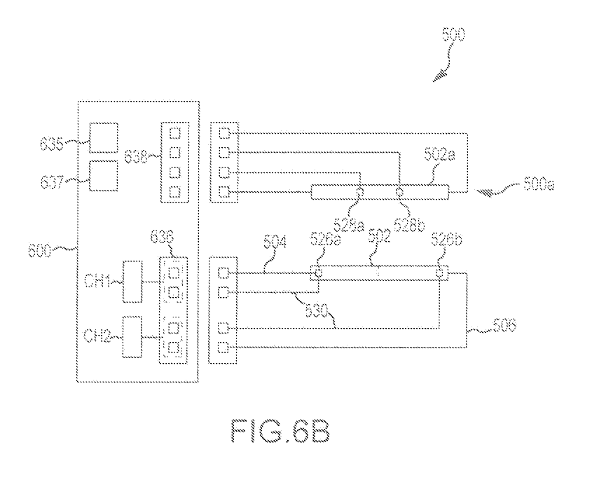

According to some embodiments, a treatment system may include or be coupled in operative association with an electrosurgical unit (ESU) that can supply and control power to an ablation assembly of the treatment system. FIGS. 5, 6A, and 6B illustrate aspects of a treatment system 500 that includes or is coupled with an ESU 600 that supplies and controls power, such RF power, during a treatment procedure. As shown here, ESU 600 includes a controller 635, a source of RF power 637 that is controlled by the controller, and a plurality of displays and buttons that are used to set the level of power supplied to one or more electrodes and the temperature at various locations on an electrode. The exemplary ESU 600 illustrated is operable in a bipolar mode, where tissue coagulation energy emitted by an electrode 502 is returned through a return electrode 502a, and a unipolar mode, where the tissue coagulation energy emitted by the electrode is returned through one or more indifferent electrodes (not shown) that are externally attached to the skin of the patient with a patch or one or more electrodes (not shown) that are positioned in the blood pool. The return electrode 502a, which in a bipolar configuration can be identical to the electrode 502, may be connected to the ESU 600 by a pair of power return lines 504a and 506a. The return electrode 502a and power return lines 504a and 506a together define a return electrode assembly 500a.

In some embodiments, return electrode 502a can be an indifferent electrode. In a bipolar configuration, an active electrode and an indifferent electrode can cooperate to help form a complete circuit of RF energy, for example when the two electrodes are placed across an anatomical feature such as the atria or other patient tissue. Energy can travel from the active electrode through the tissue to the indifferent electrode. An active electrode can be temperature controlled, and can be coupled with one or more RF wires and one or more thermocouples. An indifferent electrode can provide a return path, optionally as a single wire, operating as a ground. In use, energy passing through the electrodes can raise the temperature of the intervening tissue, for example tissue which is secured between two clamp mechanisms. In turn, the heated tissue can raise the temperature of the electrodes. In some cases, active electrodes, indifferent electrodes, or both, can be cooled with internal cooling mechanisms.

In some instances, a treatment system may include multiple active electrodes along a length of a clamp. Each active electrode can be coupled with an RF wire that supplied energy to the electrode, and two thermocouple pairs. A thermocouple pair can include two wires joined by a thermocouple, and the thermocouple can be attached to the electrode, for example at an end portion of the electrode. The thermocouple pair can be used to monitor the temperature of the electrode, or a portion of the electrode. In some embodiments, an electrode is coupled with two thermocouple pairs, and the highest of the two temperatures sensed by the thermocouple pairs can be used to control RF energy delivery to the electrode.

ESU 600 can be provided with a power output connector 636 and a pair of return connectors 638. The electrode 502 is connected to the power output connector 636 by way of the power supply lines 504 and 506 and a power connector 540, while the return electrode 502a is connected to one of the return connectors 638 by way of the power return lines 504a and 506a and a return connector 542. In some cases, the ESU output and return connectors 636 and 638 have different shapes to avoid confusion and the power and return connectors 540 and 542 are correspondingly shaped. For example, power connector 540 may have a circular shape corresponding to an ESU power output connector 636 having a circular shape, and return connector 542 may have a rectangular shape corresponding to an ESU return connector 638 having a rectangular shape. Signals from the temperature sensors 526a/526b and 528a/528b can transmitted to the ESU 600 by way of the signal lines 530 and the power connector 540.

ESU 600 can be configured to individually power and control a plurality of electrodes. In some cases, the electrodes may be about 10 mm in length. Optionally, a bipolar clamp configuration may include two 32 mm active electrodes and one 70 mm electrode. Such individually powered or controlled configurations may be referred to as providing “multi-channel control.” In some cases, ESU 600 can include up to 8 channels, or more. ESU 600 can also be configured to individually power and control two or more portions of a single electrode as well as two or more portions of each of a plurality of electrodes during a lesion formation procedure. Electrode 502 as shown here can be divided into two portions for power control purposes. The electrode portion connected to the power supply line 504 on one side of the dash line in FIG. 6A and the electrode portion connected to the power supply line 506 on the other side of the dash line. According to some embodiments, the dash line does not represent a physical division and the electrode 502 is a continuous, unitary structure. Electrode 502 can be placed adjacent to tissue and power to one portion can be controlled by control channel CH1 and power to the other portion is controlled by control channel CH2. The power can be, although not necessarily, supplied to both portions simultaneously.

According to some embodiments, the level of power supplied to the electrode 502 by way of the power supply line 504 may be controlled based on the temperatures sensed by the temperature sensors 526a/526b, while the level of power supplied to the electrode 502 by way of the power supply line 506 may be controlled based on the temperatures sensed by the temperature sensors 528a/528b. In one exemplary control scheme, the level of power supplied to the electrode 502 by way of the power supply line 504 can be controlled based on the highest of the two temperatures sensed by the temperature sensors 526a/526b, while the level of power supplied to the electrode 502 by way of the power supply line 506 can be controlled based on the highest of the two temperatures sensed by the temperature sensors 528a/528b.

The amount of power required to coagulate tissue typically ranges from 5 to 150 w. Aspects of suitable temperature sensors and power control schemes that are based on sensed temperatures are disclosed in U.S. Pat. Nos. 5,456,682, 5,582,609 and 5,755,715, the contents of which are incorporated herein by reference.

The actual number and location of the temperature sensors may be varied in order to suit particular applications. As illustrated for example in FIG. 6B, the temperature sensors 528a and 528b may be located on the return electrode 502a in certain bipolar implementations. Optionally, the power control scheme can be the same in that the level of power supplied to the electrode 502 by way of the power supply line 504 can be controlled based on the temperatures sensed by the temperature sensors 526a/526b, while the level of power supplied to the electrode 502 by way of the power supply line 506 can be controlled based on the temperatures sensed the temperature sensors 528a/528b.

According to some embodiments, a plurality of spaced electrodes can be provided that operate in a unipolar mode. Each of the electrodes can be connected to a respective pair of power supply lines and include its own set of temperature sensors. Each of the electrodes on a surgical probe can be divided into portions for power control purposes, and the level of power supplied to some electrode portions by way of power supply lines can be controlled based on the temperatures sensed by certain temperature sensors, while the level of power supplied to other electrode portions by way of power supply lines can be controlled based on the temperatures sensed by certain other temperature sensors.

As used herein, the term “clamp” or “jaw” includes, but is not limited to, clamps, jaws, clips, forceps, hemostats, and any other surgical device that includes a pair of opposable clamp members that hold tissue, at least one of which is movable relative to the other. In some instances, the clamp members are connected to a scissors-like arrangement including a pair of handle supporting arms that are pivotably connected to one another. The clamp members can be secured to one end of the arms and the handles can be secured to the other end. The clamp members can come together as the handles move toward one another. Certain clamps that are particularly useful in minimally invasive procedures also include a pair of handles and a pair of clamp members. In some cases, the clamp members and handles are not mounted on the opposite ends of the same arm. Instead, the handles can be carried by one end of an elongate housing and the clamp members are carried by the other. A suitable mechanical linkage located within the housing can cause the clamp members to move relative to one another in response to movement of the handles.

According to some embodiments, the treatment systems and methods described herein may be used in conjunction or combined with aspects of other medical systems and methods such as those described in U.S. Patent Application Nos. 60/337,070 filed Dec. 4, 2001; 10/272,446 filed Oct. 15, 2002; 10/310,675 filed Dec. 4, 2002; 10/410,618 filed Apr. 8, 2003; 11/148,611 filed Jun. 8, 2005; 60/939,201 filed May 21, 2007; 61/015,472 filed Dec. 20, 2007; 61/051,975, filed May 9, 2008; 12/124,743 filed May 21, 2008; 12/124,766 filed May 21, 2008; 12/255,076 filed Oct. 21, 2008; 12/273,938 filed Nov. 19, 2008; 12/339,331 filed Dec. 19, 2008; 12/463,760 filed May 11, 2009; 61/179,564 filed May 19, 2009; 61/231,613 filed Aug. 5, 2009; and 61/241,297 filed Sep. 10, 2009. The entire content of each of these filings is incorporated herein by reference for all purposes.

Relatedly, in some instances, the treatment systems and methods described herein may include elements or aspects of the medical systems and methods discussed in U.S. Patent Application Nos. 60/337,070 filed Dec. 4, 2001; 10/080,374 filed Feb. 19, 2002; 10/255,025 filed Sep. 24, 2002; 10/272,446 filed Oct. 15, 2002; 10/310,675 filed Dec. 4, 2002; 10/410,618 filed Apr. 8, 2003; 11/067,535 filed Feb. 25, 2005; 11/148,611 filed Jun. 8, 2005; 60/939,201 filed May 21, 2007; 61/015,472 filed Dec. 20, 2007; 61/051,975, filed May 9, 2008; 12/124,743 filed May 21, 2008; 12/124,766 filed May 21, 2008; 12/255,076 filed Oct. 21, 2008; 12/273,938 filed Nov. 19, 2008; 12/339,331 filed Dec. 19, 2008; 12/463,760 filed May 11, 2009; 61/179,564 filed May 19, 2009; 61/231,613 filed Aug. 5, 2009; and 61/241,297 filed Sep. 10, 2009. The entire content of each of these filings is incorporated herein by reference for all purposes.

Further, according to some embodiments, the treatment systems and methods described herein may be used in conjunction or combined with aspects of other medical systems and methods such as those described in U.S. patent application Ser. No. 09/268,556 filed Mar. 15, 1999; 10/272,541 filed Oct. 15, 2002; 60/431,628 filed Dec. 6, 2002; 10/727,144 filed Dec. 2, 2003; 10/731,683 filed Dec. 8, 2003; 11/186,149 filed Jul. 20, 2005; 11/753,720 filed May 25, 2007; 60/939,201 filed May 21, 2007; 61/015,472 filed Dec. 20, 2007; 12/576,607 filed Oct. 15, 2009; 61/288,031 filed Dec. 18, 2009; 12/688,618 filed Jan. 15, 2010; and 12/781,077 filed May 17, 2010. The entire content of each of these filings is incorporated herein by reference for all purposes.

While the exemplary embodiments have been described in some detail, by way of example and for clarity of understanding, those of skill in the art will recognize that a variety of modification, adaptations, and changes may be employed. Hence, the scope of the present invention should be limited solely by the claims.

What is claimed is:

1. A clamp assembly for ablating tissue, comprising:
a first jaw;
an ablation element mounted on the first jaw;
a second jaw;
a sensing element mounted on the second jaw, wherein the sensing element is configured to assess transmurality of a lesion created by the ablation element; and
a connection mechanism that connects the ablation element and the sensing element to a connector for connecting the ablation element and the sensing element to an ablation system.

2. A clamp assembly as in claim 1, wherein the connection mechanism comprises one or more wires.

3. A clamp assembly as in claim 1, wherein the sensing element comprises a temperature sensor.

4. A clamp assembly as in claim 1, wherein the sensing element comprises a pacing electrode.

5. A clamp assembly as in claim 1, wherein the sensing element comprises an optical sensor.

6. A clamp assembly as in claim 5, wherein the optical sensor detects tissue color change that occurs as tissues are heated to above 60° C.

7. A clamp assembly as in claim 5, wherein the optical sensor detects changes to near-field microwave that occur as tissues temperatures rise or fall.

8. A clamp assembly as in claim 1, wherein the sensing element penetrates a tissue surface of a tissue as the tissue is clamped between the first and second jaws of the clamp assembly.

9. A clamp assembly as in claim 8, wherein the sensing element comprises a temperature sensor.

10. A clamp assembly as in claim 8, wherein the sensing element comprises a pacing electrode.

11. A clamp assembly as in claim 8, wherein the sensing element comprises an electrode that senses tissue impedance.

12. A clamp assembly as in claim 1, wherein the ablation element heats tissue using radiofrequency energy.

13. A clamp assembly as in claim 1, wherein the ablation element heats tissue using microwave energy.

14. A clamp assembly as in claim 1, wherein the ablation element heats tissue using ultrasonic energy.

15. A clamp assembly as in claim 1, wherein the ablation element freezes tissue to achieve ablation.

16. A clamp assembly as in claim 8, further comprising one or more ablation elements coupled with the second jaw.

17. A system for ablating tissue, comprising:
an ablation clamp comprising a first jaw, a second jaw, one or more ablation elements mounted on the first jaw, and a sensing element mounted on the second jaw that assesses transmurality of a lesion created by the ablation element;
an ablation control system for controlling an ablation process provided by the ablation clamp; and
means for connecting the ablating element and the sensing element with the ablation control system.

18. A system as in claim 17, wherein the ablation control system stops the ablation process when the sensing element carried by the second jaw indicates that a transmural lesion has been created.

19. A system as in claim 17, wherein the ablation control system stops the ablation process if a predetermined maximum time limit has been reached.

20. A clamp assembly for ablating tissue, comprising:
a first jaw;
an ablation element mounted on the first jaw;
a second jaw;
a sensing element mounted on the second jaw, wherein the sensing element comprises a temperature sensor, a pacing element, or an optical sensor, wherein the sensing element penetrates a tissue surface of a tissue as the tissue is clamped between the first and second jaws of the clamp assembly, and wherein the sensing element is configured to assess transmurality of a lesion created by the ablation element; and
a connection mechanism that connects the ablation element and the sensing element to a connector for connecting the ablation element and the sensing element to an ablation system.
